(19) United States
(12) Patent Application Publication
(10) Pub. No.: US 2020/0060609 A1
(43) Pub. Date: Feb. 27, 2020

(54) METHOD AND DEVICE FOR ASCERTAINING A SKIN CONDITION

(71) Applicant: Henkel AG & Co. KGaA, Duesseldorf (DE)

(72) Inventors: ANDREAS BOCK, Neuss (DE); THOMAS WELSS, Duesseldorf (DE); CLAUDIA HUNDEIKER, Meerbusch (DE); MARIANNE WALDMANN-LAUE, Monheim (DE)

(73) Assignee: Henkel AG & Co. KGaA, Duesseldorf (DE)

(21) Appl. No.: 16/493,107

(22) PCT Filed: Mar. 5, 2018
(86) PCT No.: PCT/EP2018/055354

§ 371 Date: Sep. 11, 2019

(30) Foreign Application Priority Data
Mar. 17, 2017 (DE) 10 2017 204 493.4

(51) Int. Cl. A61B 5/00; A61B 5/145; A61B 5/1477; G16H 40/63

(52) U.S. Cl. CPC A61B 5/443; A61B 5/14546; G16H 40/63; A61B 5/6898; A61B 5/486; A61B 5/1477

(57) ABSTRACT

The present disclosure relates to a method for ascertaining the condition of the skin including: ascertaining a trace element content of skin, including: arranging a plurality of electrodes over a skin area, wherein the plurality of electrodes includes at least one first electrode and one second electrode, wherein the first electrode contacts a selection layer arranged on the skin area and the second electrode contacts the skin area, wherein only the trace element, in the event of contact with the selection layer, is suitable for generating a signal which is dependent on the concentration of the trace element and can be measured between the first and the second electrode; measuring the signal dependent on the concentration of the trace element; comparing the measured signal with reference signals for known trace element contents of skin; and ascertaining the condition of the skin on the basis of the ascertained trace element content.

### CROSS-REFERENCE TO RELATED APPLICATION

This application is a U.S. National-Stage entry under 35 U.S.C. § 371 based on International Application No. PCT/EP2018/055354, filed Mar. 5, 2018, which was published under PCT Article 21(2) and which claims priority to German Application No. 10 2017 204 493.4, filed Mar. 17, 2017, which are all hereby incorporated in their entirety by reference.

### TECHNICAL FIELD

Various embodiments relate generally to a method and device for determining the condition of the skin, a method and a device for detecting a content of trace elements in the skin, and a method for determining a cosmetic skin treatment recommendation.

### BACKGROUND

Herein, trace elements for humans are understood to mean essential chemical elements which occur in mass fractions of less than about 50 mg/kg in the organism and, because of its mode of action, additionally iron, in spite of its higher mass fraction.

During the course of aging, skin can lose trace elements which may be important for the skin physiology. For example, important enzymes of the skin may require these for, or as, what are known as co-factors, without which the enzyme cannot be active.

For care especially of skin deficient in trace elements, various special cosmetic products for the face and/or the body may be available. However, the efficacy of cosmetic products may greatly depend on an individual physiology of the skin.

Without expert dermatological or cosmetic advice, it can be difficult for a user to determine his/her individual skin condition and to ascertain the cosmetics suitable for his/her skin condition. A trace element content in the blood of the user may additionally be inappropriate for ascertaining a skin trace element content, for example since the epidermis, as the outermost barrier layer of the skin, is not connected to the bloodstream.

If the user nevertheless consumes cosmetic products, it might be practically impossible to trace a treatment success because the user, for example at home, has a lack of opportunities to assess a treatment result in a standardized and objective manner.

This can make it difficult for the consumer to assess individual efficacy of a cosmetic, which can lead to a detrimental effect on the motivation to perform an appropriate cosmetic treatment, for example in the longer term. This may even be the case if a cosmetic product were suitable for achieving an objectively verifiable desired effect.

In many areas of daily life there has for some time been a trend toward personalized programs that can respond to individual requirements and needs, for example in the nutritional or health sector, but also in the field of personalized cosmetics. This may allow a user to find cosmetic products in a targeted manner and/or to receive care instructions that are tailored to individual needs of his/her skin, and thus allow a particularly high level of efficacy.

### BRIEF SUMMARY

In various exemplary embodiments a device for ascertaining the condition of the skin of a user with inclusion of an ascertained trace element content can be provided. Ascertaining the content of trace elements on or in the skin and continuous checking of changes can provide the user with additional benefits and individualizable treatment in various exemplary embodiments.

In various exemplary embodiments, in order to ascertain the condition of the skin or the trace element content of the skin, a sensor can be used, which detects the content of essential trace elements selected from the group including chromium, cobalt, iron, copper, manganese, molybdenum, selenium, silicon, zinc, tin, vanadium, nickel, arsenic, barium, strontium, aluminum and combinations thereof in the skin (more specifically, a sensor measured value can be detected and on this basis the trace element content for one of the trace elements can be ascertained). For this, a special device can be used, which may be connectable to a smartphone, a tablet, smart mirror or another data processing device (for example a computer) or may be integrated therein.

In various exemplary embodiments the detected trace element content can be ascertained by employing an app or another software and can be provided in the form of a value (for example with arbitrary units), as a graphical representation and/or acoustic message.

In various exemplary embodiments a status with regard to the content of trace elements can be recorded over a longer period of time on the basis of the trace element content.

In various exemplary embodiments the result of an imbalance can be described on the basis of the trace element content.

In various exemplary embodiments an individualized cosmetic treatment to optimize the state can be recommended on the basis of the trace element content.

In various exemplary embodiments selective chemical sensors can be used as a sensor. In various exemplary embodiments ion-selective electrodes (ISE) can be used as a sensor, for example chip-based chalcogenide glass ISE. In various exemplary embodiments other suitable sensors and techniques can be used to quantify trace elements of the skin.

The device in various exemplary embodiments can be formed so as to be convenient and small (for example portable). The device can be used on a (living) user, for example when the user is at home, at a point of sale in cosmetic stores, at a dermatologist, and/or in a beauty salon. In various exemplary embodiments the device can include a smartphone, tablet, iPad or a similar electronic circuit device (for example a data processing device and/or a data transmission device), and can be an integrated device or an auxiliary device, which for example is mountable on and/or connectable to the electronic circuit device (for example connectable by employing a data connection). The integrated device or the auxiliary device (as applicable even the entire device) can be of such a size that it can be carried without difficulty in a handbag or trouser pocket, for example with an area of less than about 36 cm² and with a thickness of less than about 2 cm.

In various exemplary embodiments the trace element content of the user can be determined by employing the device for ascertaining the condition of the skin with the use of electrodes. In various exemplary embodiments a plurality of electrodes can be brought into contact with the skin of the user in order to determine the trace element content of the user, wherein one of the electrodes (also referred to as first electrode) can contact the skin indirectly by employing a selection layer. In various exemplary embodiments the at least one further electrode (also referred to a second electrode) can contact the skin directly. In various exemplary embodiments a solvent (for example water) can be arranged between the plurality of electrodes and the skin, for example in the form of a solvent layer. The trace elements present in the skin can pass at least partially into the solvent in various exemplary embodiments and are detectable there by employing the plurality of electrodes (for example one trace element each per plurality, for example pair or triplet, of electrodes).

In various exemplary embodiments the selection layer can include at least one reactive component, which may be configured to react with the trace element so as to release electrons or ions, wherein the released electrons or ions may be configured to reach the first electrode in order to generate a signal dependent on a concentration of the trace element.

In various exemplary embodiments the selection layer may include a partially permeable barrier layer, which may be configured to be permeable only to the trace element, in such a way that only the trace element can reach the first electrode after having passed through the barrier layer in order to generate the signal dependent on the concentration of the trace element.

In various exemplary embodiments the signal ascertained with use of the electrodes can be compared with reference signals for known trace element contents, and on the basis thereof a trace element content of the user can be ascertained (wherein the comparison can also be performed in such a way that the signal is converted into a trace element content of the user by employing a relationship ascertained on the basis of reference signals and reference data). The reference data in various exemplary embodiments may have been ascertained beforehand, for example in laboratory tests.

In various exemplary embodiments the comparison of the signal with the reference signal and/or the ascertainment of the trace element content therefrom by employing the electronic circuit device can be performed (for example directly) for example by employing an app or another software program which is operated by the electronic circuit device. The electronic circuit device for example can be a smartphone, tablet, an iPad, or the like.

In various exemplary embodiments the comparison of the signal with the reference signal and/or the ascertainment of the trace element content therefrom can be performed indirectly by employing the electronic circuit device, for example in that the electronic circuit device provides a (for example contactless) data communication connection, and, by employing the data communication connection, provides the signal and/or the result of the comparison of the signal with the reference signal to an external data processing device (for example a cloud) and receives, from the external data processing device, the trace element content ascertained by employing the signal and/or the result of the comparison.

The ascertained trace element content in various exemplary embodiments can be provided to the user in any form, for example selectable by the user, for example as a numerical value (a main trace element content measured value), a graphical representation (for example a representation of the ascertained value in relation to a total value range ranging from very deficient in trace element, through normal, to very rich in trace element), an acoustic message, or the like.

In various exemplary embodiments the device may also be configured to ascertain the condition of the skin on the basis of the ascertained trace element content. In a case in which the trace element content is or has been ascertained for only one skin area, the condition of the skin ascertained on the basis thereof can be ascertained as a uniform condition of the skin for the one ascertained trace element content, for example skin very deficient in the trace element in the case of a very low trace element content, skin very rich in the trace element in the case of a very high trace element content, normal skin in the case of a normal trace element content, etc., with an arbitrary number of gradations (for example therebetween). In a case in which the trace element content is or has been ascertained for a number of skin areas, the condition of the skin ascertained on the basis thereof can be ascertained in various exemplary embodiments as a single condition of the skin for the plurality of ascertained trace element contents, for example as combination skin with areas of skin deficient in the trace element and areas of skin rich in the trace element (for example in the case of a low trace element content ascertained for at least one first skin area and a high trace element content ascertained for at least one second skin area).

In various exemplary embodiments the condition of the skin can be specified numerically, for example as a sum value, whereby a bolding (for example of the representation of the result) can be increased.

In particular in a case in which the trace element content is ascertained for a plurality of skin areas, the provision of the condition of the skin to the user can include a graphical representation, for example a display, for example by employing a display device. In the graphical representation the ascertained trace element content of the plurality of areas can be displayed by a coding on the basis of the trace element content in a representation of the user (for example a schematic representation or on a photo of the user). For example, areas of different trace element content can be shown by different colors and/or patterns in a schematic representation of the face of the user, for example areas with a normal trace element content can be shown in green, those with a low trace element content can be shown red, and those with a high trace element content can be shown blue, or the like. In another example a photo of the user, for example a digital photo, which shows the face of the user, can be superimposed by different patterns, for example a dot pattern for areas with a high trace element content and a line pattern for areas with a low trace element content, wherein areas of normal skin may remain un-patterned, or the like.

In various exemplary embodiments when graphically representing the condition of the skin, for example the trace element content of the at least one skin area, for example the plurality of skin areas, the trace element content can be represented only for the area/the areas for which the trace element content has been ascertained. In various exemplary embodiments an area for which an ascertained trace element content is displayed can be extrapolated beyond the skin area for which the trace element content has been ascertained, for example with inclusion of typical skin trace element issuance patterns.

In various exemplary embodiments, information as to where the area/the areas is/are can be provided to the electronic circuit device, for example by employing the user, for example by employing an input device (for example tapping a touch-sensitive display on which a schematic or photographic representation of the user is displayed, by inputting text by way of a keyboard, or any other suitable input possibility).

In various exemplary embodiments the display device can be part of the device, for example a display of a smartphone, tablet or iPad. In various exemplary embodiments the display device can be coupled to the device, for example by employing a (for example wireless) data connection.

Additionally or alternatively, in various exemplary embodiments a quality value can be assigned to the ascertained condition of the skin, with a high quality value being assigned for normal/healthy skin and a low quality value being assigned for skin that is too deficient in the trace element/too rich in the trace element.

In various exemplary embodiments the electronic circuit device can be configured to ascertain the condition of the skin itself, i.e. directly, for example by employing a software, for example an app, that can be installed on the electronic circuit device.

In various exemplary embodiments the electronic circuit device can be configured to ascertain the condition of the skin indirectly, for example in that the electronic circuit device prompts the ascertainment of the condition of the skin by employing the data processing device, for example by employing software, for example an app, which can be installed on the external data processing device, for example in a manner similar to that described above for the ascertainment of the trace element content.

In various exemplary embodiments the software/app for ascertaining the trace element content can be the same software/app for ascertaining the condition of the skin. In various exemplary embodiments, for example in the case that the trace element content is ascertained by employing the external data processing device, whereas the condition of the skin is ascertained by the electronic circuit device, or vice versa, different software/apps can be used in order to ascertain the trace element content and in order to ascertain the condition of the skin.

In various exemplary embodiments a method and a device for ascertaining the condition of the skin can be provided. For this, a trace element content can be ascertained in various exemplary embodiments. The device, the method, and/or the condition of the skin ascertained by employing the device and/or the method can be used in order to ascertain the requirements of the skin of the user.

In various exemplary embodiments it should be made possible for a user to find cosmetic products in a targeted manner and/or to receive care advice that can be tailored to individual needs of the skin of the user.

In various exemplary embodiments a software, for example an app, which for example can be installed on a portable data processing device (for example a smartphone, tablet, iPad or the like), can be used in order to ascertain the trace element content and/or in order to ascertain the condition of the skin. A plurality of electrodes, which can be designed in such a way that only the trace element whose content in the skin is to be ascertained is suitable for generating, between the electrodes, a measurable signal dependent on a concentration of the trace element, can be connected to the data processing device or integrated therein. The trace element content of the skin can be ascertained by employing the plurality of electrodes. Furthermore, the condition of the skin can be ascertained for example on the basis of a trace element content measurement for an individual skin area or on the basis of a plurality of trace element content measurements for different skin areas, for example skin rich in trace element, normal skin, skin deficient in trace element, or combination skin, which includes both areas of skin deficient in trace element and areas of skin rich in trace element.

In various exemplary embodiments the ascertained trace element content and/or the ascertained condition of the skin can be displayed or communicated by employing the portable data processing device, for example by employing the software/app, in the form of values (for example with arbitrary units), as a verbal message, as a graphical representation (for example as a schematic pictorial representation including skin areas rich in trace element/deficient in trace element/normal skin), or the like.

It may be possible, in an embodiment, for the electronic circuit device and the portable data processing device to be identical to one another.

In alternative embodiments of the present disclosure the ascertained trace element content and/or the ascertained condition of the skin can be displayed by employing a smart mirror, for example by employing the software/app, as a graphical representation (for example as a schematic pictorial representation, which includes skin areas rich in trace elements/deficient in trace elements/normal skin), or the like.

In various exemplary embodiments at least one cosmetic product can be recommended to the user on the basis of the ascertained condition of the skin. The cosmetic product can be suitable for obtaining or improving the condition of the skin of the user (for example for normalizing a trace element content). In various exemplary embodiments the cosmetic products may include a cream, lotion, an ointment, an oil, an emulsion, gel, a soap, mask, an ampule with an auxiliary care product, a face lotion, a serum, a spray, or the like. The cosmetic product, besides being suitable for obtaining or improving the condition of the skin of the user, can also have further properties, for example nourishing and/or cleansing properties.

In addition, food and/or dietary supplements that are suitable for normalizing the trace element content and which contain one or more trace elements of which there is a deficiency in the skin of the user, at least in some skin areas, can be recommended to the user.

In various exemplary embodiments product recommendations for cosmetic products suited individually to the user and/or individual care advice can be derived on the basis of the ascertained trace element content and/or on the basis of the ascertained condition of the skin. The product recommendations and/or care advice can be provided for example by employing a software, for example an app. For example, it can be recommended for the user to apply a cosmetic product which includes at least one trace element for which the ascertained trace element content of the skin of the user demonstrates a deficiency for at least one skin area to the main area demonstrating the deficiency, for example to apply a trace-element-containing cream there.

In various exemplary embodiments the software/app, which ascertains the trace element content and/or the condition of the skin, can be the same as that ascertained by the product recommendation and/or the care advice. In various exemplary embodiments different software programs/apps can be used for some of the various processes or all of the various processes (ascertaining the trace element content, ascertaining the condition of the skin, ascertaining a product recommendation, ascertaining care advice).

In various exemplary embodiments a treatment success of a cosmetic treatment, the objective of which can be to positively influence the ascertained trace element content or the ascertained condition of the skin, can be monitored. In various exemplary embodiments the software/app can make it possible to check and/or track the results by employing a representation (for example a graphical representation) of the measurement result over time.

In various exemplary embodiments, when ascertaining the product and care recommendations, information regarding a general state of health, the condition of the skin, dietary habits and further behaviors of the user (for example time spent daily outdoors/in the sun/in water (for example amongst other things in mineral-rich health spas, which can also be rich in trace elements), smoking habits, dietary habits, etc.) can also be used, for example by employing the software/app. In various exemplary embodiments this information can be asked of the user by employing the software/app.

In various exemplary embodiments data from the literature can form the basis of the assessment of a suitability of a cosmetic product and/or care advice for caring for skin of a given condition.

In various exemplary embodiments a quality value can be assigned to each condition of the skin of a plurality of conditions of the skin. A cosmetic product and/or care advice for a condition of the skin can be assessed as suitable if it is anticipated, for example on account of data from the literature, test results or values gained from experience (which for example can be updated continuously, for example also during use of the software/the app by the user, with new values gained from the experience of other users; see below), that with (for example regular) use of the cosmetic product and/or adherence to the care advice, the condition of the skin of the user will be maintained or will change to a condition of the skin having a higher (i.e. better) quality value.

In various exemplary embodiments the assessment of the suitability of a cosmetic product for improving a condition of the skin can be confirmed or modified by employing an inclusion of values gained from the experience of further users with the same or a similar condition of the skin, for example values gained from experience with regard to a treatment success. The further users, in addition to the same or similar condition of the skin, may also have the same or a similar profile in respect of age, gender and/or behavior. The user can thus now always receive an optimal recommendation.

In various exemplary embodiments a checking and a tracking of the efficacy of a cosmetic treatment in an objective and standardized manner can be made possible. The cosmetic treatment in various exemplary embodiments can have the objective of normalizing a trace element content.

In various exemplary embodiments the efficacy of a (for example cosmetic) treatment can be better understood, and therefore the selection of an individually suitable product can be simplified.

In various exemplary embodiments the motivation of a user to perform a cosmetic treatment in the longer term can be increased, for example by employing a comparison with other users for example by employing information regarding treatment successes provided by the other users.

In various exemplary embodiments a method for ascertaining the condition of the skin is provided. The method can include: ascertaining a trace element content of skin, including: for at least one skin area of the user, arranging a plurality of electrodes over the skin area, wherein the plurality of electrodes includes at least one first electrode and one second electrode, wherein the first electrode contacts a selection layer arranged on the skin area and the second electrode contacts the skin area, wherein the selection layer is designed in a selective manner for a trace element such that only the trace element, in the event of contact with the selection layer, is suitable for generating a signal which is dependent on the concentration of the trace element and can be measured between the first and the second electrode; measuring the signal dependent on the concentration of the trace element; comparing the measured signal with reference signals for known trace element contents of skin; and ascertaining the condition of the skin on the basis of the ascertained trace element content of the skin.

In various exemplary embodiments the selection layer can include at least one reactive component, which can be configured to react with the trace element so as to release electrons or ions, and wherein the released electrons or ions can be configured to reach the first electrode in order to generate the signal dependent on the concentration of the trace element.

In various exemplary embodiments the selection layer can include a partially permeable barrier layer, which can be configured to be permeable only for the trace element, in such a way that only the trace element can reach the first electrode after having passed through the barrier layer in order to generate the signal dependent on the concentration of the trace element.

In various exemplary embodiments the method, prior to the arrangement of the plurality of electrodes over the at least one skin area, can also include a wetting of the at least one skin area by employing a solvent.

In various exemplary embodiments the solvent can include water.

In various exemplary embodiments the trace element can include one from a group of trace elements, wherein the group can include chromium, cobalt, iron, copper, manganese, molybdenum, selenium, silicon, zinc, tin, vanadium, nickel, arsenic, barium, strontium, aluminum. The trace element may be selected from the group including chromium, cobalt, iron, copper, manganese, molybdenum, selenium, silicon, zinc and combinations.

In various exemplary embodiments the at least one skin area can include a plurality of skin areas of the user.

In various exemplary embodiments the plurality of electrodes can be part of a portable device.

In various exemplary embodiments the portable device can include a smartphone, tablet, or an iPad, or can be attached to a smartphone, tablet or an iPad.

In various exemplary embodiments the comparison of the measured signal with reference signals for known trace element contents of skin and the ascertainment of the condition of the skin on the basis of the ascertained trace element content of the skin can be performed by employing an app.

In various exemplary embodiments a method for ascertaining a cosmetic skin treatment recommendation is provided. The method can include ascertaining the condition of the skin in accordance with various exemplary embodiments and ascertaining the cosmetic product and/or care advice on the basis of the ascertained condition of the skin and a database, wherein the database can include a plurality of conditions of the skin and a plurality of assigned cosmetic products and/or pieces of care advice, wherein each condition of the skin of the plurality of conditions of the skin can be assigned at least one suitable cosmetic product and/or at least one piece of care advice.

In various exemplary embodiments each condition of the skin of the plurality of conditions of the skin can be assigned a quality value, and a cosmetic product and/or the care advice can be suitable for the condition of the skin of the plurality of conditions of the skin if, on the basis of stored values gained from experience with the cosmetic product, an improvement or maintenance of the quality value of the condition of the skin is to be expected.

In various exemplary embodiments the method may also include an updating of the database on the basis of new values gained from experience of a plurality of users.

In various exemplary embodiments the ascertained condition of the skin can be a condition of deficiency in respect of the trace element, and the cosmetic product can include the trace element.

In various exemplary embodiments the ascertainment of the content of a trace element of a skin area and/or the ascertainment of the condition of the skin can include transmitting the signal, a result of the comparison and/or the ascertained content of the trace element of the skin area to an external data processing device and receiving the content of the trace element of the skin area and/or of the condition of the skin.

In various exemplary embodiments a device for ascertaining the condition of the skin is provided. The device can include: a plurality of electrodes, wherein the plurality of electrodes includes at least one first electrode and one second electrode; a selection layer, wherein the selection layer is designed in a selective manner for a trace element such that only the trace element is suitable, in the event of contact with the selection layer, for generating a signal which is dependent on the concentration of the trace element and can be measured between the first and the second electrode, wherein the first electrode, the second electrode and the selection layer are designed in such a way that the plurality of electrodes and the selection layer can be arranged over a skin area of a user in such a way that the selection layer contacts the skin area, the first electrode contacts the selection layer, and the second electrode contacts the skin area; and an electronic circuit device, which is designed to measure the signal dependent on the concentration of the trace element, to ascertain the trace element content on the basis of a comparison between the measured signal and reference signals for known trace element contents of skin, and to ascertain the condition of the skin on the basis of the trace element content.

In various exemplary embodiments the selection layer can include at least one reactive component, which is designed to react with the trace element so as to release electrons or ions, wherein the released electrons or ions can be configured to reach the first electrode in order to generate the signal dependent on the concentration of the trace element.

In various exemplary embodiments the selection layer may include a partially permeable barrier layer, which may be configured to be permeable only to the trace element, in such a way that only the trace element can reach the first electrode after having passed through the barrier layer in order to generate the signal dependent on the concentration of the trace element.

In various exemplary embodiments the electronic circuit device and a plurality of electrodes can form an integrated unit.

In various exemplary embodiments the integrated unit may also include the selection layer.

In various exemplary embodiments the plurality of electrodes can be part of a portable device.

In various exemplary embodiments the portable device can include a smartphone, tablet or an iPad.

In various exemplary embodiments the portable device may also include a data exchange device for wireless data exchange.

### BRIEF DESCRIPTION OF THE DRAWINGS

In the drawings, similar reference signs usually refer to the same parts in all different views, wherein for the sake of clarity it is sometimes the case that not all parts corresponding to one another have been provided with reference signs in all figures. For differentiation, parts that are the same or similar can be provided, in addition to a common reference sign, also with a subsequent number or a lowercase letter. The drawings are not necessarily intended to provide a true-to-scale representation, but instead the emphasis lies in illustrating the principles of the present disclosure. Various embodiments of the present disclosure will be described in the following description with reference to the following drawings, in which:

FIG. 1A and FIG. 1B show a device for ascertaining the condition of the skin in accordance with various exemplary embodiments and use of the device;

FIG. 2A and FIG. 2B show a device for ascertaining the condition of the skin in accordance with various exemplary embodiments and use of the device;

FIG. 3A and FIG. 3B show a device for ascertaining the condition of the skin in accordance with various exemplary embodiments;

FIG. 4 shows skin regions for application of a method and a device for ascertaining the condition of the skin in accordance with various exemplary embodiments;

FIG. 5 shows a flow diagram for illustrating a method for ascertaining a skin treatment recommendation in accordance with various exemplary embodiments;

FIG. 6 shows a flow diagram of a method for ascertaining the condition of the skin in accordance with various exemplary embodiments; and

FIG. 7 shows a flow diagram of a method for ascertaining a cosmetic skin treatment recommendation in accordance with various exemplary embodiments.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The word “exemplary” is used herein in the sense of “by way of example, for instance, or for illustration purposes”. All embodiments or designs described herein as “exemplary” are not necessarily meant as being preferred or advantageous over other embodiments or designs.

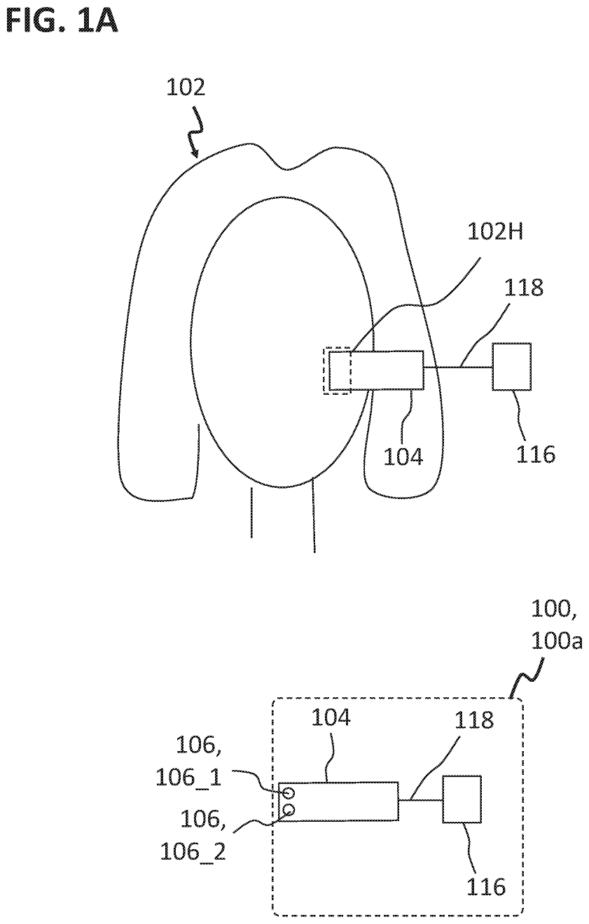

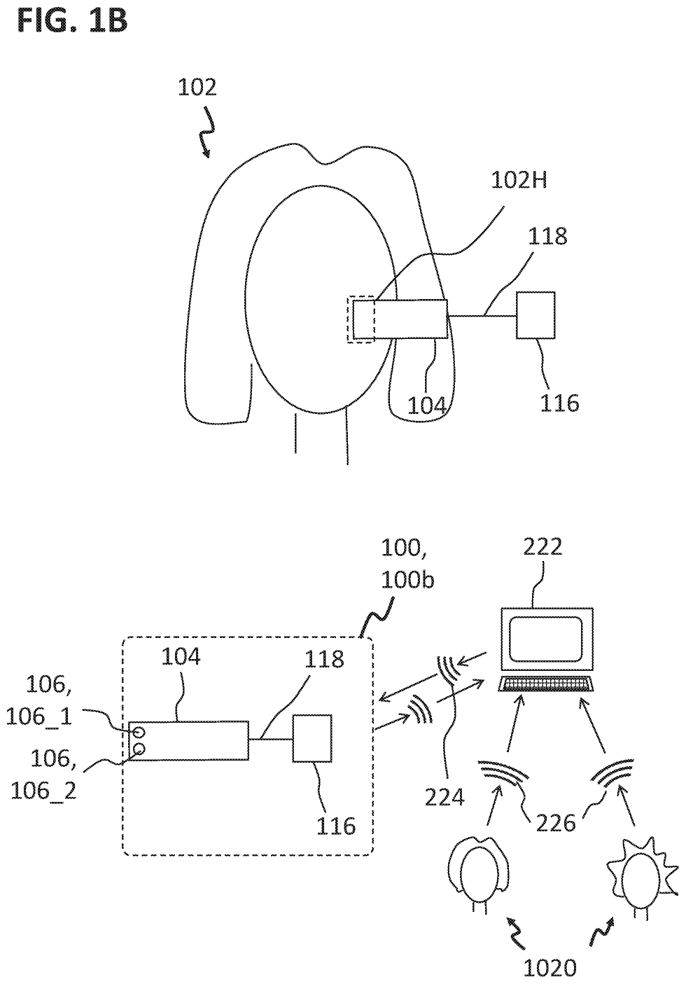

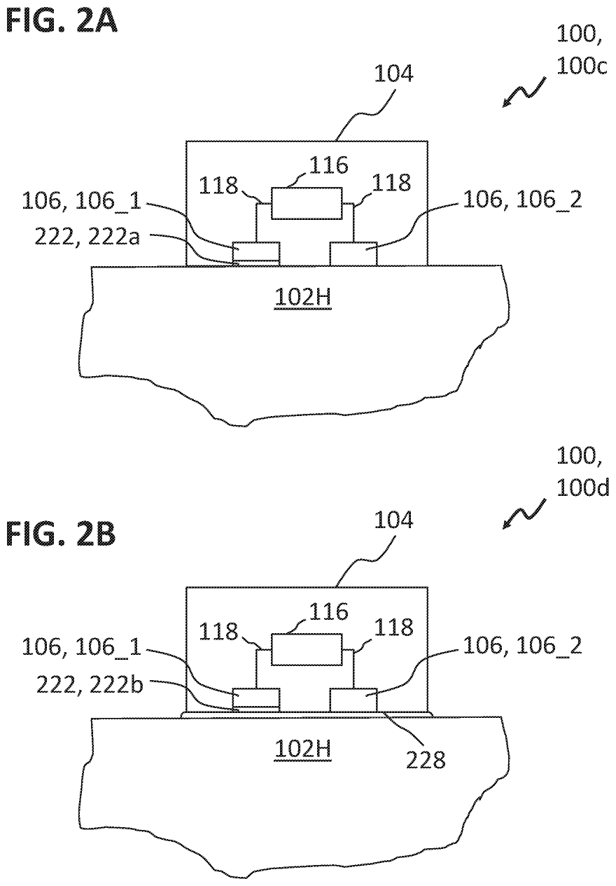

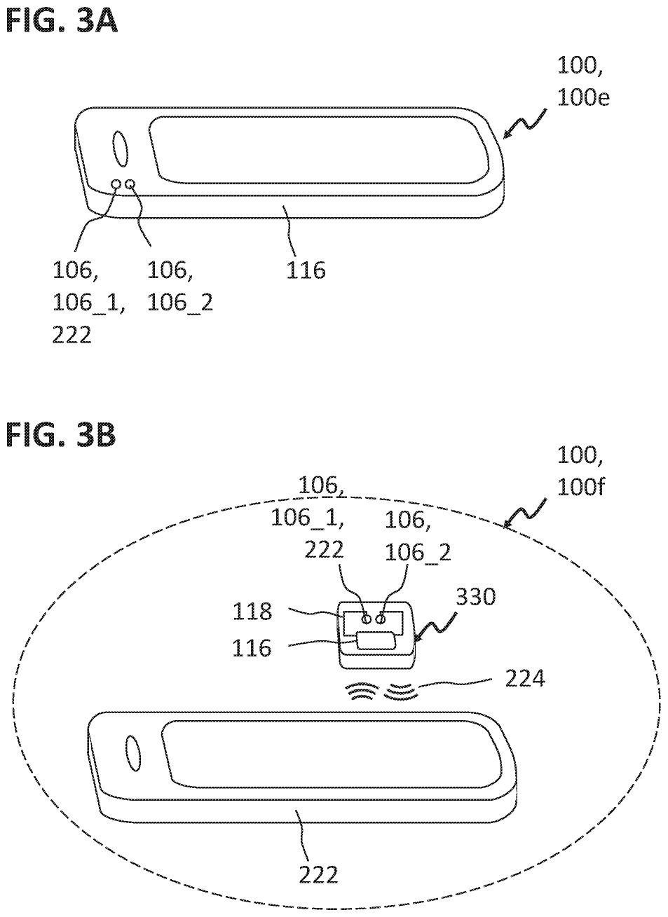

FIG. 1A, FIG. 1B, FIG. 2A, FIG. 2B, FIG. 3A and FIG. 3B show a device 100 (100a, 100b, 100c, 100d, 100e or 100f) for ascertaining the condition of the skin of a user 102 in accordance with various exemplary embodiments.

In various exemplary embodiments the device 100 for ascertaining the condition of the skin can include a plurality of electrodes 106, wherein the plurality of electrodes can include at least one first electrode 106_1 and one second electrode 106_2. In various exemplary embodiments the device 100 can also include a selection layer 222, wherein the selection layer 222 can be designed in a selective manner for a (for example single) trace element, such that only the (for example the single) trace element, in the event of contact with the selection layer 222, is suitable for generating a signal that is dependent on a concentration of the trace element and can be measured between the first and the second electrode 106_1 or 106_2, wherein the first electrode 106_1, the second electrode 106_2 and the selection layer 222 can be designed in such a way that the plurality of electrodes 106 and the selection layer 222 can be arranged over a skin area 102H of a user 102 in such a way that the selection layer 222 contacts the skin area 102H, the first electrode 106_1 contacts the selection layer 222, and the second electrode 106_2 contacts the skin area 102H. In other words, as is shown in particular in FIG. 2A and FIG. 2B, the first electrode 106_1 can contact the skin area 102H of the user 102 indirectly by employing the selection layer 222, whereas the second electrode 106_2 contacts the skin area of the user 102 directly (see FIG. 2A), or the first electrode 106_1 can contact the skin area 102H of the user 102 indirectly by employing the selection layer 222 and a solvent (for example a solvent layer) 228, whereas the second electrode 106_2 can contact the skin area of the user 102 indirectly by employing the solvent (for example a solvent layer) 228 (see FIG. 2B).

The solvent, in various exemplary embodiments, can include water or another solvent in which trace elements from the skin of the user 102 can dissolve at least in part and which can be suitable for cooperating with the selection layer 222 and the electrodes 106 in such a way that a signal dependent on the concentration of the trace element in the skin is generated between the electrodes 106.

In various exemplary embodiments the signal generated between the electrodes 106 can be measured as a voltage, resistance or current, for example in a manner that is fundamentally known.

In various exemplary embodiments the selection layer 222a (see FIG. 2A) can include at least one reactive component, which can be designed to react with the trace element that is to be examined so as to release electrons or ions, wherein the released electrons or ions can be designed to reach the first electrode 106_1 in order to generate the signal dependent on the concentration of the trace element that is to be examined.

In other words, in various exemplary embodiments, the trace element (for example as part of a molecule, as an ion, or as an atom) can infiltrate the selection layer 222a from the skin area 102H of the user directly (or indirectly by employing the solvent layer 228, not shown) and can react there (for example chemically) with the at least one reactive component, for example in the form of a redox reaction, and in so doing can release at least one electron and/or at least one ion. The electron or the ion can move towards the first electrode 106_1, where it can generate a signal in combination with the second electrode 106_2. With a higher concentration of the trace element in the skin area 102H, more reactions with the reactive component can be triggered, and thus more electrons or ions can be released, which can migrate to the first electrode 106_1, so that a stronger signal between the two electrodes 106_1, 106_2 can be triggered, i.e. the generated signal can be dependent on the concentration of the trace element in the skin area 102H. The relationship between the signal and the trace element content of the skin and/or reference signals with assigned trace element content values (for example in table form) can be ascertained in various exemplary embodiments beforehand, for example in laboratory tests.

In various exemplary embodiments the selection layer 222 can include a partially permeable barrier layer 222b (see FIG. 2B), which can be designed to be permeable only for the trace element that is to be examined (for example as part of a molecule, as an ion, or as an atom), in such a way that only the trace element that is to be examined can reach the first electrode 106_1 after having passed through the barrier layer 222b. The trace elements reaching the first electrode 106_1 can generate a signal between the first electrode 106_1 and the second electrode 106_2. With a higher concentration of the trace element in the skin of the user 102, more trace elements can pass through the partially permeable barrier layer 222b, so that the generated signal can be dependent on the concentration of the trace element.

In various exemplary embodiments the plurality of electrodes 106 can include for example an electrode pair, for example formed of a working electrode (the first electrode 106_1) and counter electrode (the second electrode 106_2), or for example an electrode triplet, for example formed of working electrode (the first electrode 106_1), counter electrode (the second electrode 106_2) and reference electrode (not shown).

In various exemplary embodiments the trace element can include one from a group of trace elements, wherein the group comprises chromium, cobalt, iron, copper, manganese, molybdenum, selenium, silicon, zinc, tin, vanadium, nickel, arsenic, barium, strontium, aluminum, and combinations thereof. Here, the plurality of electrodes 106 can be designed to ascertain the concentration merely of a single one of the trace elements. In various exemplary embodiments (not shown), a number of pluralities of electrodes 106, which can differ from one another at least in respect of the selection layer 222, can be provided in order to ascertain a plurality of different trace element contents.

In various exemplary embodiments, for example a first plurality of electrodes can be designed such that a selection layer which can be arranged between the first electrode and the skin can react with a first trace element or is permeable for same, whereas a second plurality of electrodes can be designed such that a selection layer which can be arranged between the first electrode of the second plurality of electrodes and the skin can react with a second trace element or is permeable for same.

In various exemplary embodiments the first plurality of electrodes and the second plurality of electrodes can be part of a single device. In various exemplary embodiments the first plurality of electrodes and the second plurality of electrodes can be arranged in or on the device in such a way that the skin of the user 102 can be contacted simultaneously by both pluralities of electrodes. In various exemplary embodiments the first plurality of electrodes and the second plurality of electrodes can be arranged in or on the device in such a way that the skin of the user 102 can be contacted sequentially by both pluralities of electrodes.

In various exemplary embodiments more than two pluralities of electrodes can be provided in the device and can be designed to detect further trace elements.

In various exemplary embodiments the device 100 for ascertaining the condition of the skin may also include an electronic circuit device 116, which can be designed to measure the signal dependent on the concentration of the trace element, to ascertain the trace element content on the basis of a comparison between the measured signal and reference signals for known trace element contents of the skin, and to ascertain the condition of the skin on the basis of the trace element content.

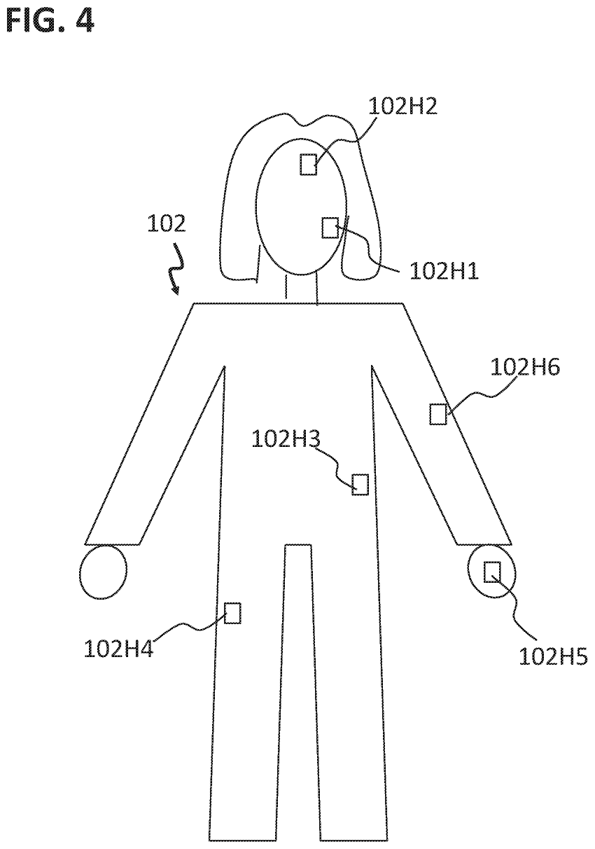

The condition of the skin can be ascertained in various exemplary embodiments by ascertaining a trace element content for at least one skin area 102H of the user 102 and by ascertaining the condition of the skin on the basis thereof. As is shown as main area 102H in FIG. 1A and FIG. 1B, the skin area 102H in various exemplary embodiments can be part of the skin of the face of the user 102. Alternatively or additionally, as shown in FIG. 4, which shows skin regions 102H for application of a method and a device for ascertaining the condition of the skin in accordance with various exemplary embodiments, another or further skin area 102, for example on an arm (main area 102H6), a leg (main area 102H4), a torso (area 102H3) and/or a hand (area 102H5) of a user, can be used to ascertain the condition of the skin of this skin area.

In various exemplary embodiments the electrode 106 can be coupled to the electronic circuit device 116, for example by employing a (wired or wireless) data connection 118.

The electronic circuit device 16 in various exemplary embodiments can be configured, as described above, to ascertain a trace element content of the skin of the user 102 (with measurement of a plurality of skin areas 102H, the trace element content of the skin area in question), for example a very high, moderately high, normal, moderately low, or very low trace element content, by employing comparison of the signal generated between the electrodes 106. In various exemplary embodiments the trace element content can be described or quantified differently, for example as a trace element content number (for example with an arbitrary unit) or the like.

With use of the trace element content (or the plurality of trace element contents), the condition of the skin of the user 102 can be ascertained in various exemplary embodiments as described above.

In various exemplary embodiments, the condition of the skin may correspond to the ascertained trace element content with (in respect of its value) substantially only one ascertained trace element content or with uniform ascertained trace element contents for a plurality of different skin areas: for example, in the case of an ascertained very high trace element content for the single skin area, or all or almost all skin areas, the ascertained condition of the skin can be very rich in trace element, and in the case of an ascertained very low trace element content for the single skin area, or all or almost all skin areas, the ascertained condition of the skin can be very deficient in trace element, etc.

In various exemplary embodiments, with (in respect of its value) uniform ascertained trace element contents for a plurality of different skin areas, the condition of the skin can be ascertained as combination skin (i.e. a skin that includes both areas deficient in trace element and areas rich in trace element), wherein, depending on the trace element content of the areas deficient in trace element and/or rich in trace element, different types of combination skin can be ascertained, for example as described above.

The device 100 for ascertaining the condition of the skin can be a portable device 100 in accordance with various exemplary embodiments.

The electronic circuit device 116 can include a processor and a memory in various exemplary embodiments. The electronic circuit device 116 in various exemplary embodiments can be coupled to the plurality of electrodes 106, for example in such a way that it is possible to detect a signal, which is generated between the plurality of electrodes 106, for example by employing a data connection 118. The data connection 118 can include, for example, a data cable, an internally conductive connection and/or a wireless transmission, for example by employing WLAN, Thread, ZigBee or Bluetooth. The electronic circuit device 116 can be, for example, a smartphone, an iPad, tablet, laptop, smart mirror, or the like.

In various exemplary embodiments (for example as shown in FIG. 3A and FIG. 3B) the plurality of electrodes 106 and the electronic circuit device 116 can form a portable integrated unit.

For example, the plurality of electrodes 106 and the electronic circuit device 116 as a portable integrated unit can form the skin examination device 100, for example as shown in FIG. 3A for a smartphone with electrodes 106 integrated in a housing of the smartphone.

In another example the plurality of electrodes 106 and the electronic circuit device 116 can form a portable integrated unit 330 (see FIG. 3B), which can be coupled to an external data processing device 222, for example a smartphone or the like, for example by employing a wireless data transmission 224, for example as described elsewhere herein (wherein alternatively another form of the data transmission can be used, for example a wired form). A combination of the integrated unit 330 and the external data processing device 222 in various exemplary embodiments can form the device 100f for ascertaining the condition of the skin.

In various exemplary embodiments the integrated device 100e, 100f can be of such a size that it can be carried in a handbag or trouser pocket. For example, the integrated device can have an area of less than about 36 cm² and a thickness of less than about 2 cm.

The electronic circuit device 116 can be configured in various exemplary embodiments to ascertain a trace element content on the basis of the measured electrode signal. In various exemplary embodiments the electronic circuit device 116 can also be configured to ascertain the condition of the skin on the basis of the at least one trace element content. In various exemplary embodiments the electronic circuit device 116 can be designed to ascertain the trace element content of the skin and/or the condition of the skin itself, i.e. directly.

In various exemplary embodiments a software, for example an app, which for example can be installed on a portable data processing device, wherein the data processing device for example can be the electronic circuit device 116, as shown for example in FIG. 3A, or an additional, for example external, data processing device 222, as shown for example in FIG. 3B, can be used in order to ascertain the trace element content.

In various exemplary embodiments the trace element content or the condition of the skin can be ascertained time-dependently (for example multiple times daily, daily, weekly, or with any other time dependency) and/or in different ambient conditions (for example in an environment of high or low air humidity, in the cold, or in the heat, etc.).

In various exemplary embodiments calibration measurements (also referred to as reference measurements), which for example can be carried out in a laboratory and the results of which can be stored in the data processing device, can be used for example as part of the software/app in order to assign a signal measurable between the electrodes, for example as described above (for example a voltage, current, or a resistance) and/or the at least one ascertained trace element content to a condition of the skin.

In various exemplary embodiments the ascertainment of the trace element content, for example as described above, can be carried out directly by employing the portable electronic circuit device 116, for example by employing the software/app, which can be installed on the portable electronic circuit device 116.

In various exemplary embodiments the trace element content can be provided by employing the electronic circuit device, for example by employing the software/app. The trace element content can be communicated, for example represented, for example as a value (for example with arbitrary units), as a verbal message, as a graphical representation, or the like, for example by employing being displayed, for example on a screen of the portable electronic circuit device 116.

In various exemplary embodiments the ascertainment of the trace element content, for example as described above, can be performed indirectly by employing the portable electronic circuit device 116, for example in that the electronic circuit device 116 prompts the ascertainment of the trace element content by employing an external data processing device 222. The electronic circuit device 116 in various exemplary embodiments can include a relatively simple electronic circuit device, which for example can be designed to measure substantially merely the signal between the electrodes 106 (as applicable, to compare it with the reference signals) and to transmit it to the external data processing device 222. In various exemplary embodiments the electronic circuit device 116 can include a more powerful, versatile circuit device (for example a smartphone, tablet or an iPad), which can be configured not only to measure the signal between the electrodes 106 (and, as applicable, to compare it with the reference signals) and to transmit it to the external data processing device 222, but additionally may be configured to carry out various further functions, for example to ask the user 102 to make inputs and to store these, to represent results, to provide a photo of the user 102 for displaying the results (for example by employing a camera) and to process the same, etc.

In various exemplary embodiments the measurement result of the electrode signal and/or the comparison result from the portable electronic circuit device 116, as shown in FIG. 1B, can be provided, for example transmitted, to the external data processing device 222, for example a central computer, a smart mirror or a cloud, which for example can have a greater computing power and/or a greater storage capacity than the electronic circuit device 116. The external data processing device 222 can be configured to ascertain the trace element content from the provided data, for example the signal or the result of the comparison, for example by employing a software, for example an app, for example as described above. In various exemplary embodiments the electronic circuit device 116 can also be configured to receive the trace element content from the external data processing device 222 and to provide it, for example as described above. For data exchange with the external data processing device 222, the electronic circuit device 116 in various exemplary embodiments can include a device for wireless data transmission, for example by employing WLAN, Thread, ZigBee or Bluetooth. In various exemplary embodiments the electronic circuit device 216 can be configured to exchange the data with the external data processing device 222 by employing a wired connection.

In various exemplary embodiments the electronic circuit device 116 can be designed to ascertain the condition of the skin on the basis of the ascertained trace element content.

In various exemplary embodiments the electronic circuit device 116 can be configured to ascertain the condition of the skin itself, i.e. directly, for example by employing a software, for example an app, which can be installed on the portable electronic circuit device 116.

In various exemplary embodiments the electronic circuit device 116 can be designed to ascertain the condition of the skin indirectly, for example in that the electronic circuit device 116 prompts the ascertainment of the condition of the skin by employing the external data processing device, for example by employing a software, for example an app, which can be installed on the external data processing device 222, for example in a manner similar to that described above for the ascertainment of the trace element content.

In various exemplary embodiments the software/app used to ascertain the trace element content of the skin can be the same software/app used to ascertain the condition of the skin. In various exemplary embodiments, for example in a case in which the trace element content of the skin is ascertained by employing the external data processing device 222, whereas the condition of the skin is ascertained by the portable electronic circuit device 116, or vice versa, different software/apps can be used in order to ascertain the trace element content and in order to ascertain the condition of the skin.

In various exemplary embodiments the ascertainment of the condition of the skin can include a comparison of the at least one ascertained trace element content with entries in a database, which can include a plurality of conditions of the skin each with at least one assigned trace element content. In various exemplary embodiments, in the event that more than one trace element content is assigned to a condition of the skin, the skin area for which the trace element content in the database was ascertained is additionally recorded in the database. In such a case, when ascertaining the condition of the skin by employing a plurality of trace element contents, the skin areas for which each trace element content was ascertained can be taken into consideration, provided the skin areas are known, for example were specified by the user, or can be assumed, for example because the user was instructed to perform the trace element measurement in certain skin areas (possibly in a certain order).

In various exemplary embodiments the database can be stored in the portable electronic circuit device 116 and/or in the external data processing device 222.

When ascertaining the condition of the skin, the condition of the skin for which the trace element content (or trace element contents) demonstrates the smallest deviations from the trace element content(s) assigned to the condition of the skin in the database can be ascertained as the condition of the skin of the user.

In various exemplary embodiments the device 100 can be configured to provide the user 102 with the ascertained condition of the skin, for example by employing the software/app. The condition of the skin can be communicated, for example represented, for example as a value (for example with arbitrary units), as a verbal message, as a graphical representation, or the like, for example by employing being displayed, for example on a screen of the portable electronic circuit device 116.

In particular in a case in which the trace element content is ascertained for a plurality of skin areas, the provision of the condition of the skin to the user can include a graphical representation, for example by being displayed, for example by employing a display device (for example a screen/display). In various exemplary embodiments a representation of a body or a body area (for example of the face) can be used for this, for example as a symbolic representation or as a photo of the user 102. The representation can be superimposed by an illustration of the trace element content or the condition of the skin, for example by employing different symbols/markings/hatching for areas of different trace element content or condition of the skin.

For example, in a case in which the trace element content is ascertained for a plurality of skin areas, the provision of the condition of the skin to the user can include a graphical representation on a smart mirror. For this, a real-time representation of a body or of a body area (for example of the face) on/in the smart mirror can be used in various exemplary embodiments. The representation can be superimposed by an illustration of the trace element content or the condition of the skin, for example by employing different symbols/markings/hatching for areas of different trace element content or condition of the skin.

In various exemplary embodiments the device 100 can also be configured to ascertain a cosmetic skin treatment recommendation on the basis of the ascertained condition of the skin and to provide this to the user 102.

In various exemplary embodiments at least one suitable cosmetic product and/or at least one piece of care advice can be assigned to each of the plurality of conditions of the skin. The assignments might have been ascertained for example experimentally, for example during the course of laboratory tests.

In various exemplary embodiments data from the literature can form the basis of the assessment of a suitability of a cosmetic product and/or care advice for caring for skin of a given condition.

In various exemplary embodiments a quality value can be assigned to each of the conditions of the skin. A cosmetic product and/or a piece of care advice can be assessed as being suitable for a condition of the skin if it is expected, for example on account of data from the literature, test results or values gained from experience, that, with (for example regular) use of the cosmetic product and/or adherence to the care advice, the condition of the skin of the user 102 will be maintained or will change to a condition of the skin having a higher quality value.

In various exemplary embodiments, as shown in FIG. 1B, an assessment of a suitability of a cosmetic product to improve the condition of the skin can be confirmed or modified by employing an inclusion of values gained from the experience of further users 1020 having the same or a similar condition of the skin, for example from values gained from experience in respect of a treatment success. The values gained from experience can be provided by the further users, for example to the external data processing device 222, for example by employing a wireless data transmission 226. Alternatively, a transmission of the data by employing cables can also be used. The database in the external data processing device 222 and/or in the portable electronic circuit device 116 can be updated on the basis of the values gained from experience. It can thus be made possible that the user 102 always receives an optimal recommendation.

In various exemplary embodiments, when ascertaining the product and/or care recommendations, information regarding a general state of health, the condition of the skin, dietary habits and further behaviors of the user (for example daily time spent outside/in the sun/in water (for example in water containing a trace element), smoking habits, dietary habits, etc.) can also be used, for example by employing the software/app installed on the portable electronic circuit device 116 and/or on the external data processing device 222. The information, in various exemplary embodiments, can be asked of the user 102 by employing the portable electronic circuit device 116, and can be input by the user into the electronic circuit device 116, for example by employing a keyboard, in the form of a voice message, as a selection from a menu presented by the portable electronic circuit device 116, or the like.

For example, if the user 102 provides the additional information that he/she spends a lot of time in water or outdoors, when providing the product and/or care advice recommendation on the basis of the condition of the skin of the user 102, for example the assigned cosmetic products recommended to the user 102 can be limited to those that not only are not suitable for his/her condition of the skin, but in addition for example are water-resistant and/or are provided with a UV filter, and/or in the event that an oversupply of the skin with trace elements is determined and information is given stating that the user 102 frequently visits health spas, care advice can be provided to limit the frequency/length of the time spent in the spa water.

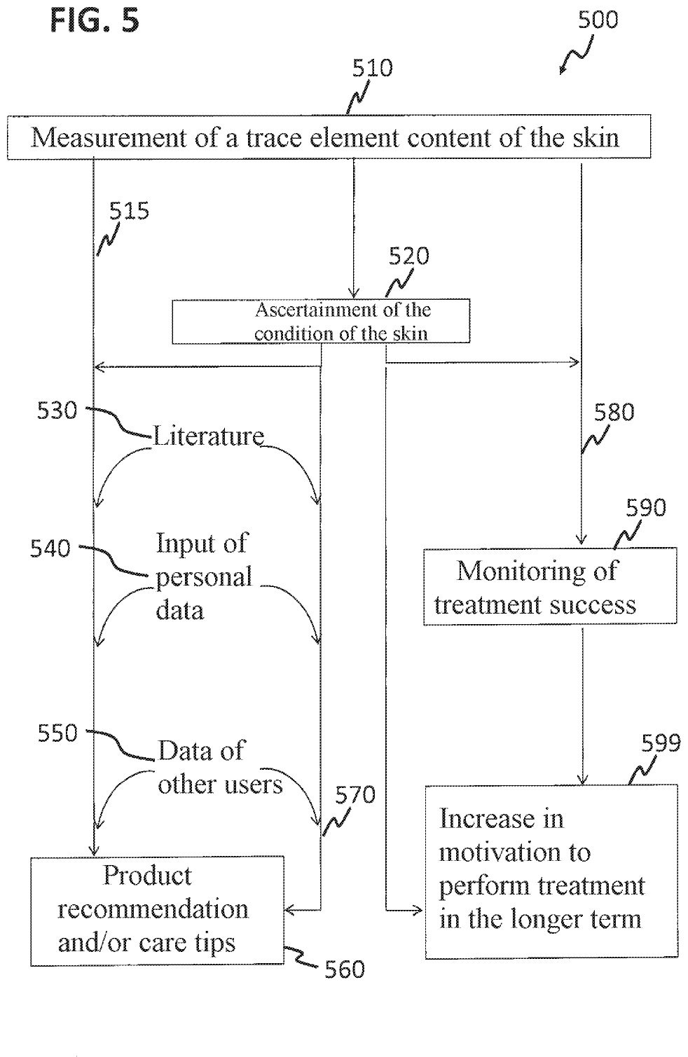

In various exemplary embodiments the product recommendation and/or the care advice can be provided to the user 102 for example by employing the electronic circuit device 116, for example by employing the screen of the electronic circuit device 116. For example, the graphical representation 500 from FIG. 5 can be supplemented by (for example text) messages (not shown), for example the ascertained product recommendations and/or care advice, for example “apply cream A in the hatched area”, or the like.

FIG. 5 shows a diagram 500 for illustrating a method for ascertaining a cosmetic product for skin treatment in accordance with various exemplary embodiments. FIG. 5 can explain substantially those processes that are described elsewhere in conjunction with the method for ascertaining a cosmetic product for skin treatment in accordance with various exemplary embodiments.

The method, in accordance with various exemplary embodiments, can include ascertaining a trace element content of the skin (at 510), for example as described above.

In various exemplary embodiments the method may also include ascertaining the condition of the skin (also referred to as skin type) (at 520), for example as described above.

On the basis of the ascertained condition of the skin and/or on the basis of the ascertained trace element content, a product or care tip recommendation can be ascertained (at 560). In various exemplary embodiments (shown as path 570), only the condition of the skin ascertained on the basis of the trace element content may be used.

In various exemplary embodiments (shown as path 515), data from the literature (denoted by 530), the personal data (for example as described above, denoted by 540), and/or data of other users, for example their values gained from experience (denoted as 550) can be added additionally to the trace element content.

In various exemplary embodiments, as shown at the path 580, a treatment success can also be monitored (at 590) by employing ascertaining the trace element content (at 510). This may be expedient for example in particular during and after a cosmetic treatment on the basis of the product recommendation (at 560). A treatment success demonstrated by employing the method for ascertaining the condition of the skin on the basis of objective values can increase the motivation of the user (at 599).

In various exemplary embodiments, as described above, product recommendations for cosmetic products suited individually for the user 102 and/or individual care advice can be derived on the basis of the ascertained trace element content and/or on the basis of the ascertained condition of the skin. The product recommendations and/or care advice can be provided for example by employing a software, for example an app. In addition, a recommended cosmetic product can be offered for sale to the user, and the user can arrange for the purchase via an input. Besides the purchase of cosmetic products, further information in addition to the purchase can also be provided to the user. This further information can relate to more detailed individual care advice. The software/app program for example receives the request that the user wishes to purchase the recommended cosmetic product, stores the request and/or transmits the request to a trading company which sells the products. The user is requested by the software/app to input his/her personal data (address, bank information, shipment preference, etc.). Alternatively, the user can be provided with information as to where (for example drugstore, beauty salon, perfume store, pharmacy, etc.) he/she can purchase the recommended product.

In various exemplary embodiments the user may be advised to use cosmetic products that will be produced tailored for the user, and an ordering process can be initiated, for example by loading the website of a manufacturer of tailored cosmetic products.

Customers always desire a product tailored individually to their requirements. This product may be a product produced especially for the customer or what is known as a “mass customized” product. In the case of a “mass customized” product an individualization can be achieved by varying just a few features of a product that are considered by the customer, however, to be critical. In an embodiment, these “mass customized” products are based on the concept of modularization, that is to say the product can be composed individually from various modules/building blocks.

There are often numerous dependencies between the many different features/ingredients of the product, and these can be expressed as “dos” and “don'ts”. In order to attain a unique product definition, it may be advantageous for the ordering process to proceed with the aid of a product configurator. This configurator helps the customer choose the features/ingredients and advises him/her of the admissible/inadmissible combinations of features, wherein the latter then cannot be selected.

In the case of products for the skin, the relevant product features comprise in particular the chemical ingredients of the product, the physical properties of the product, and the formulation of the product. For example, with the aid of a product configurator, the selection of chemically and/or physically incompatible ingredients or the selection of ingredients that are unsuitable for the ascertained condition of the skin can be avoided. Conversely, the selection of ingredients that are suitable for the ascertained condition of the skin can be specified or proposed by the product configurator.

In various exemplary embodiments it may be recommended to visit a dermatologist or a beautician. In various exemplary embodiments a booking process can be initiated directly by the software/app which ascertains the trace element content of the skin and/or the condition of the skin and/or the product recommendation and/or the care advice. To this end, for example, the contact data of dermatologists and/or beauticians can be stored in the software/app and can be displayed to the user. In addition, the selection can be limited via filters, for example postcode. In various exemplary embodiments an appointment can be booked directly via the software/app. Alternatively, an appointment with a dermatologist and/or a beautician can be booked via a separate software/app, for example Treatwell.

In various exemplary embodiments the software/app which ascertains the trace element content of the skin and/or the condition of the skin can be the same as that which ascertains the product recommendation and/or the care advice and/or initiates the ordering process for a recommended product. In various exemplary embodiments different software programs/apps can be used for part of the various processes or all various processes (ascertaining the trace element content, ascertaining the condition of the skin, ascertaining a product recommendation, ascertaining care advice, initiating and ordering process).

In various exemplary embodiments a treatment success of a cosmetic treatment, the objective of which may be to positively influence the ascertained trace element content of the skin or the ascertained condition of the skin can be monitored. In various exemplary embodiments the software/app can make it possible to check and/or track the results by employing a representation (for example a graphical representation) of the measurement result over time.

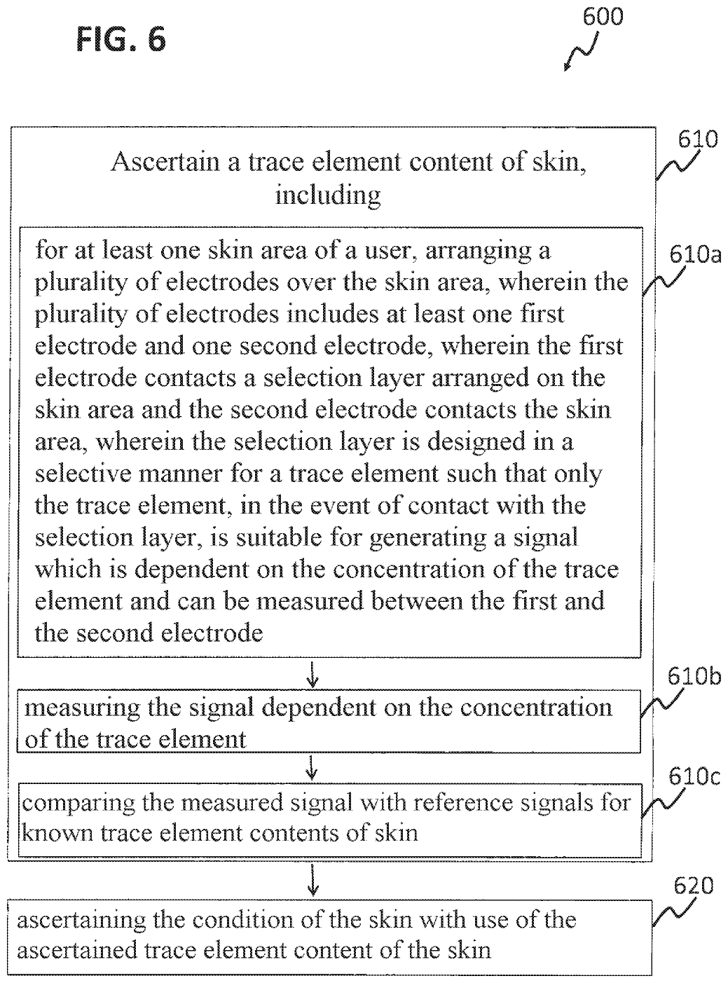

FIG. 6 shows a flow diagram 600 of a method for ascertaining the condition of the skin in accordance with various exemplary embodiments.

In various exemplary embodiments a method for ascertaining the condition of the skin is provided. The method can include ascertaining a trace element content (at 610), including: for at least one skin area of a user, arranging a plurality of electrodes over the skin area, wherein the plurality of electrodes includes at least one first electrode and one second electrode, wherein the first electrode contacts a selection layer arranged on the skin area and the second electrode contacts the skin area, wherein the selection layer is designed in a selective manner for a trace element such that only the trace element, in the event of contact with the selection layer, is suitable for generating a signal which is dependent on the concentration of the trace element and can be measured between the first and the second electrode (at 610a); measuring the signal dependent on the concentration of the trace element (at 610b); and comparing the measured signal with reference signals for known trace element contents of skin (at 610c); and ascertaining the condition of the skin with use of the ascertained trace element content of the skin (at 620).

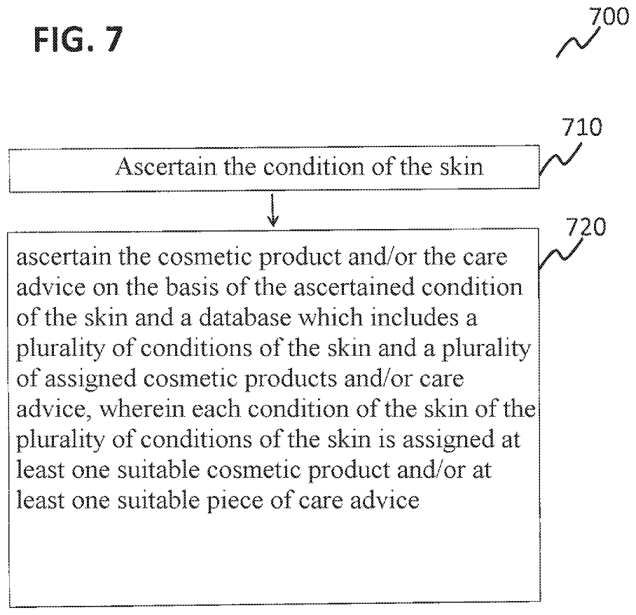

FIG. 7 shows a flow diagram 700 of a method for ascertaining a cosmetic skin treatment recommendation in accordance with various exemplary embodiments.

In various exemplary embodiments a method for ascertaining a cosmetic skin treatment recommendation is provided. The method can include: ascertaining the condition of the skin in accordance with various exemplary embodiments (at 710) and ascertaining the cosmetic product and/or the care advice on the basis of the ascertained condition of the skin and a database which includes a plurality of conditions of the skin and a plurality of assigned cosmetic products and/or pieces of care advice, wherein each condition of the skin of the plurality of conditions of the skin can be assigned at least one suitable cosmetic product and/or at least one suitable piece of care advice (at 720).

Some of the exemplary embodiments are described in conjunction with devices, and some of the exemplary embodiments are described in conjunction with methods. Further advantageous embodiments of the method will become clear from the description of the device, and vice versa.

In particular, the following devices also form the subject of the present disclosure:

A device for ascertaining the condition of the skin, including:
a plurality of electrodes, wherein the plurality of electrodes includes at least one first electrode and one second electrode;
a selection layer, wherein the selection layer is designed in a selective manner for the trace element in such a way that only the trace element is suitable, in the event of contact with the selection layer, to generate a signal which is dependent on a concentration of the trace element and can be measured between the first and the second electrode;
wherein the first electrode, the second electrode, and the selection layer are designed in such a way that the plurality of electrodes and the selection layer can be arranged over a skin area of a user in such a way that the selection layer contacts the skin area, the first electrode contacts the selection layer, and the second electrode contacts the skin area;
an electronic circuit device, which is configured to measure the signal dependent on the concentration of the trace element, to ascertain the trace element content on the basis of a comparison between the measured signal and reference signals for known trace element contents of skin, and to ascertain the condition of the skin on the basis of the trace element content.

The device as contemplated herein,
wherein the selection layer includes at least one reactive component, which is configured to react with the trace element so as to release electrons or ions, and
wherein the released electrons or ions are configured to reach the first electrode in order to generate the signal dependent on the concentration of the trace element.

The device as contemplated herein,
wherein the selection layer includes a partially permeable barrier layer, which is configured to be permeable only for the trace element, in such a way that only the trace element can reach the first electrode after having passed through the barrier layer in order to generate the signal dependent on the concentration of the trace element.

The device as contemplated herein,
wherein the electronic circuit device and the plurality of electrodes form an integrated unit.

The device as contemplated herein,
wherein the integrated unit also includes the selection layer.

The device as contemplated herein,
wherein the plurality of electrodes is part of a portable device.

The device as contemplated herein,
wherein the portable device includes a smartphone, tablet, or an iPad.

The device as contemplated herein,
wherein the portable device also includes a data exchange device for wireless data exchange.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

Listing of claims:

1. A method for ascertaining a condition of skin, comprising:
ascertaining a trace element content of the skin, comprising:
for skin area of a user, arranging a plurality of electrodes over the skin area, wherein the plurality of electrodes comprises a first electrode and a second electrode, wherein the first electrode contacts a selection layer arranged on the skin area and the second electrode contacts the skin area, wherein the selection layer is designed in a selective manner for a trace element such that only the trace element, in the event of contact with the selection layer, is suitable for generating a signal which is dependent on a concentration of the trace element and can be measured between the first and the second electrode;
measuring the signal dependent on the concentration of the trace element;
ascertaining trace element content of the skin by comparing the measured signal with reference signals for known trace element contents of skin; and
ascertaining the condition of the skin on the basis of the ascertained trace element content of the skin.

2. The method according to claim 1,
wherein the selection layer comprises a reactive component, which is configured to react with the trace element so as to release electrons or ions, and
wherein the released electrons or ions are configured to reach the first electrode in order to generate the signal dependent on the concentration of the trace element.

3. The method according to claim 1,
wherein the selection layer comprises a partially permeable barrier layer, which is configured to be permeable only for the trace element, in such a way that only the trace element can reach the first electrode after having passed through the barrier layer in order to generate the signal dependent on the concentration of the trace element.

4. The method according to claim 1, further comprising:
prior to the arrangement of the plurality of electrodes over the skin area, wetting the at least one skin area by means of a solvent.

5. The method according to claim 1, wherein the trace element comprises one from a group of trace elements, wherein the group of trace elements comprises:
chromium;
cobalt;
iron;
copper;
manganese;
molybdenum;
selenium;
silicon;
zinc;
tin;
vanadium;
nickel;
arsenic;
barium;
strontium;
aluminum; and
combinations thereof.

6. The method according to claim 1, wherein the skin area comprises a plurality of skin areas of the user.

7. The method according to claim 1,
wherein the plurality of electrodes comprises a part of a portable device.

8. The method according to claim 7,
wherein the portable device comprises a smartphone, a tablet, or an iPad, or wherein the portable device is attachable to a smartphone, a tablet, or an iPad.

9. The method according to claim 1,
wherein the comparison of the measured signal with reference signals for known trace element contents of skin and the ascertainment of the condition of the skin on the basis of the ascertained trace element content of the skin are performed by means of an electronic application on a portable device.

10. A method for ascertaining a cosmetic skin treatment recommendation, comprising: ascertaining the condition of the skin in accordance with claim 1; and
ascertaining the a cosmetic product and/or a piece of care advice on the basis of the ascertained condition of the skin and a database which comprises a plurality of conditions of the skin and a plurality of assigned cosmetic products and/or a plurality of pieces of care advice, wherein each condition of the skin of the plurality of conditions of the skin is assigned a suitable cosmetic product of the plurality of assigned cosmetic products or a suitable piece of care advice of the plurality of pieces of care advice.

11. The method according to claim 10,
wherein each condition of the skin of the plurality of conditions of the skin is assigned a quality value, and the suitable cosmetic product or the suitable piece of care advice is suitable for the condition of the skin of the plurality of conditions of the skin if an improvement or maintenance of the quality value of the condition of the skin is to be expected on the basis of stored values gained from experience with the cosmetic product.

12. The method according to claim 11, further comprising:
updating the database on the basis of new values gained from experience from a plurality of users.

13. The method according to claim 10,
wherein the ascertained condition of the skin is a condition of deficiency in respect of the trace element; and
wherein the suitable cosmetic product comprises the trace element.

14. The method according to claim 1,
wherein the ascertainment of the content of a trace element of a skin area and/or the ascertainment of the condition of the skin includes comprises a transmission of the signal, a result of the comparison and/or the ascertained content of the trace element of the skin area to an external data processing device and a receipt of the content of the trace element of the skin area and/or of the condition of the skin.

15. A device for ascertaining a condition of skin, comprising:
a plurality of electrodes, wherein the plurality of electrodes comprises a first electrode and a second electrode;
a selection layer, wherein the selection layer is designed in a selective manner for a trace element in such a way that only the trace element, in the event of contact with the selection layer, is suitable for generating a signal which is dependent on a concentration of the trace element and can be measured between the first and the second electrode;
wherein the first electrode, the second electrode and the selection layer are designed in such a way that the plurality of electrodes and the selection layer can be arranged over a skin area of a user in such a way that the selection layer contacts the skin area, the first electrode contacts the selection layer, and the second electrode contacts the skin area;
an electronic circuit device, which is designed to measure the signal dependent on the concentration of the trace element, to ascertain the a trace element content on the basis of a comparison between the measured signal and reference signals for known trace element contents of skin, and to ascertain the condition of the skin on the basis of the trace element content.
